SEQUENCE LISTING (21 sequences)

SEQ ID NO: 1 - protein, 46 aa, Homo sapiens
KMWDQEKDHLKKFNELMVMFRVRPTVLMPLWNVLGFALGAGTALLG

SEQ ID NO: 2 - protein, 6 aa, Homo sapiens
MWDQEK

SEQ ID NO: 3 - protein, 4 aa, Homo sapiens
DHLK

SEQ ID NO: 4 - protein, 9 aa, Homo sapiens
FNELMVMER

SEQ ID NO: 5 - protein, 25 aa, Homo sapiens
VRPTVLMPLWNVLGFALGAGTALLG

SEQ ID NO: 6 - protein, 10 aa, Homo sapiens
DQEKDHLKKF

SEQ ID NO: 7 - protein, 7 aa, Homo sapiens
NELMVMF

SEQ ID NO: 8 - protein, 11 aa, Homo sapiens
RVRPTVLMPLW

SEQ ID NO: 9 - protein, 5 aa, Homo sapiens
NVLGF

SEQ ID NO: 10 - protein, 10 aa, Homo sapiens
ALGAGTALLG

SEQ ID NO: 11 - protein, 13 aa, Bos taurus
LHSMKEGIHAQQK

SEQ ID NO: 12 - protein, 19 aa, Bos taurus
VPQLEIVPNSAEERLHSMK

SEQ ID NO: 13 - protein, 7 aa, Bos taurus
RLHSMKE

SEQ ID NO: 14 - protein, 5 aa, Bos taurus
LHSMK

SEQ ID NO: 15 - protein, 15 aa, Bos taurus
IVPNSAEERLHSMKE

SEQ ID NO: 16 - protein, 14 aa, Bos taurus
IVPNSAEERLHSMK

SEQ ID NO: 17 - protein, 22 aa, Bos taurus
MKLLILTCLVAVALARPKHPIK

SEQ ID NO: 18 - protein, 33 aa, Bos taurus
MKLLILTCLVAVALARPKHPIKHQGLPQEVLNE

SEQ ID NO: 19 - DNA, 42 bp, Homo sapiens
attttgagaacctctgaggaaaccatttctacagttcaagaa

SEQ ID NO: 20 - protein, 56 aa, Homo sapiens
RTSEETISTVQEKQQNISPLVRERGPQRVAAHITGTRGRSNTLSSPNSKNEKALGR

SEQ ID NO: 21 - protein, 21 aa, Homo sapiens
RTSEETISTVQDSKNEKALGR

(19) United States
(12) Patent Application Publication
(10) Pub. No.: US 2004/0002118 A1
(43) Pub. Date: Jan. 1, 2004

(54) Method for determining mass altering moiety in peptides

(76) Inventor: Zeev Smilansky, Meghar (IL)

Correspondence Address: MERCHANT & GOULD PC, P.O. BOX 2903, MINNEAPOLIS, MN, 55402-0903, US

(21) Appl. No.: 10/399,018

(22) PCT Filed: Oct. 11, 2001
(86) PCT No.: PCT/IL01/00944

(30) Foreign Application Priority Data
Oct. 11, 2000 (IL) 138946

(51) Int. Cl. G01N 33/53; G06F 19/00; G01N 33/48; G01N 33/50

(52) U.S. Cl. 435/7.1; 702/19

(57) ABSTRACT

A method is described for determining the mass of a mass altering moiety, and for identifying a cleavage altering sequence, wherein the mass altering moiety or a cleavage altering sequence is present in an assayed peptide and is absent from a corresponding database peptide, or is present in a database peptide and is absent from an assayed peptide.

### FIELD OF THE INVENTION

The present invention relates to the identification of proteins and their addition moieties by mass spectrometry, wherein mass spectrometry data are compared with sequence database, and wherein important additional information about the identified protein is obtained by usage of several proteolytic agents and careful analysis of the results. The invention further relates to a kit for identification and characterization of proteins and their addition moieties by mass spectrometry.

### BACKGROUND OF THE INVENTION

Biological mass spectrometry (MS) has become an indispensable tool for rapid protein and peptide structural analysis [WO 93/24834 (1993); U.S. Pat. No. 5,538,897 (1996); Wise M. J. et al.: Electrophoresis 18 (1997)1399-1409; Jonscher K. R. et al.: Analytical Biochemistry 244 (1997) 1-15; Perkins D. N. et al.: Electrophoresis 20 (1999) 3551-3567; Gevaret K. et al.: Electrophoresis 21 (2000) 1145-1154].

When the mass of individual molecules has to be assayed, the molecules are converted to gas-phase ions prior to detection. Ions produced in the ion source are separated in a mass analyzer according to their m/z ratio and are usually detected by a micro-channel plate. The MS data is typically recorded as a “spectrum” which displays ion abundance versus the respective m/z value.

The identification of proteins by MS is considerably simplified if the protein is already represented in a sequence database. In this case, the identity is established by correlating the experimental data with data present in the database, utilizing various algorithms for protein identification [Yates J. R. III: Electrophoresis 19 (1998) 893-900]. Two types of MS data are used for the protein identification: (i) mass spectra of a mixture of peptides derived by specific cleavage of the protein with proteolytic enzymes, called “peptide-mass fingerprinting”(PMF); or (ii) mass spectra obtained from the fragmentation pattern of an individual peptide isolated after proteolysis of the target protein, called MS/MS. An important caveat to those methods of identification is that the amino acid sequence of the protein, or the nucleotide sequence coding therefor, is contained within the database being searched.

Peptide-mass searching is a method for the identification of proteins contained in a database using an algorithm matching a set of peptide-masses generated from a protein of interest by a specific cleavage reagent (either enzymatic or chemical), with theoretical peptide masses calculated for each sequence entry in the database, assuming that the database sequence has been cleaved with the same specificity as the protein in the experiment.

Analysis of the results of peptide-mass searching experiment may not always be straightforward. Often, masses are found that do not match expected peptide masses. These “orphan” masses may be the result of one of the following: the analyzed protein is contaminated; an incorrect protein was identified (a “false-positive”), the tested protein is not identical to the database protein, but it is rather a sequence homologue of it, a splice variant of it, or a similar protein from a different species or a different strain; or the correct protein was identified by the search but the unknown masses are the result of other factors. These factors include post-translational modifications that occur in-vivo, protein cleavage (i.e. cleavage of signal peptide or preprotein), modifications that occur in-vitro during the sample preparation (i.e. chemical modifications, salt adducts, degradation, etc.) or alterations on the DNA level (i.e. polymorphism, mutation) or RNA level (i.e. RNA editing).

Thus, differences between observed and expected masses can be thought of as belonging to three different classes. Firstly, there are differences that result from the “in vivo” state of the protein, such as splice variance, post translational modifications, signal peptide cleavage, etc. Secondly, there are differences caused by the sample preparation methods, comprising salt adducts, alkylations, additional chemical moieties contaminating the sample, etc. Thirdly, there are computational problems such as incomplete databases, erroneous protein identifications, etc.

Thus, the main object of the invention is to overcome limitations of protein databases, and to allow characterization of a protein even where the specific form is not explicitly detailed in such database.

Pucci P. et al. [Biomed. Environ. Mass Spectrometry 17 (1988) 287-291] and Jaffe H. et al. [Biochemistry 37 (1998) 3931-40] deduced a phosphorylation or acetylation sites in a peptide after its digestion with a protease. Wilkins M. R. et al. [J. Mol. Biol. 289 (1999) 645-657] described several rules that help to predict which type of post-translational modifications occur at each type of amino acid, and they used these rules in localizing a modification in a protein after its digestion with one enzyme. However, deducing the type of modification or its location can be often an impossible task, in view of all the ambiguities resulting from the large amount of possible interpretations, especially in case of large peptides.

It is therefore a purpose of this invention to provide a method for the determination of the mass of a mass altering moiety which is present in an assayed peptide and absent from a corresponding database peptide, or is present in a database peptide and is absent from an assayed peptide.

It is another purpose of this invention to provide a method for the determination of the identity of a mass altering moiety.

It is also a purpose of this invention to provide a method for identifying a cleavage altering sequence which is present in an assayed peptide and is absent from a corresponding database peptide, or is present in a database peptide and is absent from an assayed peptide, which sequence comprises a cleavage site for at least one digestion agent used in the assay. It is also a purpose of this invention to identify such amino acid sequence.

It is another purpose of this invention to provide a result at a high measure of information confidence and accuracy by using two or more digestion agents.

It is a further purpose of this invention to provide a procedure that enables processing of mass spectra supplied after digesting with two or more agents, which procedure also enables automation and computerization of the method.

It is still another purpose of this invention to locate the modified site within the analyzed peptide.

It is also a purpose of this invention to identify novel modifications in proteins, hitherto not known.

### Glossary

In the description and claims to follow use will be made, at times, of a variety of terms. The meaning of such terms should be construed as follows:

Mass Altering Moiety—a moiety present in the “assayed peptide” (see below) and not present in the corresponding “database peptide” (see below) or vice versa. Typically, where the moiety is present only in the assayed peptide it is a chemical moiety added by post-translational modification in-vivo, and may be a sugar moiety, a lipophilic moiety, phosphorus moiety, methyl moiety, a moiety added by myristoylation and the like. Other modifications can occur in-vitro during the sample preparation, or might also be the result of amino-acid alterations due for example to DNA polymorphism or mutation, in case the assayed peptide and the database peptide are from the same species/strain, or due to species/strain differences between the assayed and the database proteins. Alternatively, it might be a short stretch of amino acids (one or more amino acid residues) that is different between the assayed and the database proteins, for example due to alternative splicing. By another alternative, the moiety is present in the database peptide but absent from the assayed peptide due to cleavage (for example of signal peptide), alternative splicing, species/strain differences, etc. It should be noted that where the mass alteration moiety is an amino acid sequence (added, deleted or changed in the assayed sequence), it does not change the cleavage site identified by the “digestion agent” used (see below)—i.e., it does not delete an existing cleavage site present in the database peptide and does not create a new cleavage site.

Cleavage Altering Sequence—a short stretch of amino acids (one or more) present in the assayed peptide and absent from the database peptide, or vice versa, which includes a cleavage point at least for one of the cleavage agents that are used in the assay. Thus, contrary to the situation of the mass altering moiety above, said addition, deletion or change of the amino acid changes the cleavage pattern by the digestion agent as compared to the parent protein—for example by creating a new cleavage site or by deleting an existing cleavage site.

Assayed Peptide—a sequence of amino acids present in the sample, for which not all of the chemical properties are known, such as those caused by post-translational modifications, splice variance, etc. Some of the unknown properties are to be determined by a method of the invention. The term “peptide” should be understood as referring also to a full protein or to a polypeptide.

Corresponding Database Peptide—an amino acid sequence present in a database, which can be shown to correspond to an assayed peptide by methods known in the art. These methods may include peptide-mass fingerprint or MS/MS, wherein it is determined with a high probability that a specific database peptide corresponds to the assayed peptide—albeit with differences. These differences, being the mass altering moiety, or cleavage altering sequence, are to be determined by the methods of the present invention. The meaning of “corresponding” is that the assayed peptide and the database peptide share large portions of sequences, but they may differ from each other due to post-translational modifications of the assayed peptide, or due to the fact that one is a splice variant of the other, or one is a cleaved version of the other, or they are from different species/strain, etc. The term “peptide” should be understood as referring also to a protein, and a polypeptide.

First Digestion Agent—an enzyme, or a chemical agent, which is known to cleave an amino acid sequence at a sequence-specific point (between two specific amino acid residues). Examples of the above are proteolytic enzymes, such as trypsin, Glu-C or Lys-C, or chemical cleaving agents, such as CNBr, etc.

Further Digestion Agent—a digestion agent (within the meaning specified above), which is different from the first digestion agent, i.e. which cleaves the amino acid sequence at a different sequence-specific point. The further digestion agent may be a second agent, but this term is also collectively used to denote a third, fourth, fifth or a further different digestion agent.

Fragments—subsequences of the assayed peptide (obtained by physical digestion with digestion agents) or subsequences of the database peptide obtained by theoretical digestion.

First Assayed Fragments—fragments of the assayed peptide obtained by digestion with the first digestion agent.

Further Assayed Fragments—fragments of the assayed peptide obtained by digestion with a further digestion agent.

Digestion Product—the set of all fragments obtained from digestion of an assayed peptide by a digestion agent.

Mass Spectrum of the Digestion Product—the collection of mass values obtained as a result of mass spectrometric analysis of the assayed sample.

Theoretical Fragments—fragments of the corresponding database peptide obtained by theoretical (hypothetical) cleavage of the database sequence by the digestion agent.

Threshold Value—the minimal mass difference which is significant in view of the accuracy of the mass spectrometric experiment. Usually it is either an absolute value, an example of such being 0.2 Dalton, or a relative value, for example 100 parts per million (ppm).

Mᵢ^{a}—refers to one or more mass values of each of the individual first assayed fragments present in the mass spectra of the first digestion product.

Mⱼ^{a}—refers to one or more mass values of the individual further assayed fragments obtained from the mass spectra of the further digestion product.

Mᵢ^{t}—refers to one or more mass values of the individual theoretical fragments of the corresponding database peptide, obtained by the “theoretical digestion” with the first digestion agent. Where the assayed peptide and the database peptide are completely identical, there are pairs of Mᵢ^{a}, Mᵢ^{t} with nearly identical masses, up to the machine tolerance and accuracy.

Mⱼ^{t}—refers to one or more mass values of the individual theoretical fragments of the corresponding database peptide, obtained by the “theoretical digestion” with the further digestion agent. Where the assayed peptide and the database peptide are completely identical, there are pairs of Mⱼ^{a}, Mⱼ^{t} with nearly identical masses, up to the machine tolerance and accuracy.

Dᵢ—refers to the set of all possible mass differences between the assayed Mᵢ^{a} and theoretical Mᵢ^{t}. It should be noted that each assayed mass Mᵢ^{a} is compared to all theoretical masses Mᵢ^{t}.

Dⱼ—refers to the set of all possible differences between assayed Mⱼ^{a} and theoretical Mⱼ^{t}. Each Mⱼ^{a} is compared to all theoretical masses Mⱼ^{t}.

Selected Differences D_{i′}/D_{j′}—while some of the digestion fragments of a specific database peptide present in the database are identical to the fragments of the assayed peptide (both treated by the same digestion agent), others may not be so. Differences Dᵢ and Dⱼ, which are small (as determined by a threshold value), are discarded. Selected differences D_{i′}, or D_{j′}are those wherein the mass of fragments of the assayed peptide are substantially different from the mass of any of fragments of the database peptide theoretically digested with the same digestion agent. It is noted that the term “difference between the assayed Mᵢ^{a} and theoretical Mᵢ^{t}” is used, in relevant context, also in the meaning of “absolute value of the difference between the assayed Mᵢ^{a} and theoretical Mᵢ^{t}”, especially when the comparison with the threshold value is considered.

Orphan Mᵢ^{a}, Mⱼ^{a}, Mₖ^{a}, etc—refers to one or more mass values of individual assayed fragments present in the mass spectra of a digestion product, which have no corresponding theoretical masses of the individual fragments of the corresponding database peptide.

Orphan Mᵢ^{t}, Mⱼ^{t}, Mₖ^{t}, etc—refers to one or more mass values of individual theoretical fragments of the database peptide obtained by “theoretical digestion” with a digestion agent, and which do not have corresponding masses of the assayed peptide.

First orphan region—the subset of the amino acid sequences of the database peptide, which includes all the fragments corresponding to orphan Mᵢ^{t} for said first digestion agent.

Further orphan region—the subset of the amino acid sequences of the database peptide, which includes all the fragments corresponding to orphan Mⱼ^{t} for said further digestion agent.

Peptide orphan region—the intersection of the first orphan region with all further orphan regions, thus consisting of a subset of the sequences of the database peptide that were not identified by any of the digestion agents;

Altered Fragment—a fragment of a database peptide, which was theoretically altered by addition or deletion of one or more amino acids.

Mₐₗₜ—the mass of the altered fragment.

The words Digestion and Cleavage are used interchangeably

### SUMMARY OF THE INVENTION

The present invention concerns methods for detecting, analyzing, and interpreting differences between an assayed peptide and a corresponding database peptide. This means that once a peptide was identified, with a high probability (a high “score”), as being similar to a specific corresponding database peptide in accordance with any methods known in the art (see for example the cited references: Wise et al.; Jonscher et al.; Yates et al.; Gevaret et al.; and Perkins et al.), it is possible by the method of the invention, to identify specific differences between the assayed peptide and the corresponding database peptide, including masses of the altering moieties or sequences, their identities, and location within the peptide.

The present invention relates to a method for determining the mass of a mass altering moiety, which is present in an assayed peptide and is absent from a corresponding database peptide, or is present in a database peptide and is absent from an assayed peptide, the method comprising:

(i) treating the assayed peptide with a first digestion agent to obtain a first digestion product comprising a plurality of first assayed fragments; and determining the mass spectrum of the digestion product to obtain one or more mass values of the individual first assayed fragments Mᵢ^{a};

(ii) treating the assayed peptide with a further digestion agent to obtain a further digestion product comprising a plurality of further assayed fragments; and determining the mass spectrum of the further digestion product to obtain one or more mass values of the individual further assayed fragments Mⱼ^{a};

(iii) optionally repeating step (ii) according to the number of different further digestion agents, obtaining mass values of the individual further assayed fragments Mₖ^{a}, Mᵢ^{a}, Mₘ^{a}, etc.;

(iv) optionally identifying the assayed peptide, in case of a peptide not identified earlier, by a suitable protein identification method;

(v) obtaining masses Mᵢ^{t} of the individual theoretical fragments of the database peptide corresponding to the assayed peptide, which fragments are obtained by the theoretical digestion of said database peptide with said first digestion agent;

(vi) obtaining masses Mⱼ^{t} of the individual theoretical fragments of the database peptide corresponding to the assayed peptide, which fragments are obtained by the theoretical digestion of said database peptide with said further digestion agent;

(vii) comparing each of Mᵢ^{a} with each database value Mᵢ^{t}, to obtain a plurality of differences Dᵢ=Mᵢ^{a}−Mᵢ^{t} and discarding all Dᵢ values lower than a predetermined threshold value to give a plurality of selected differences Dᵢ′;

(viii) comparing each of Mⱼ^{a} with each database value Mⱼ^{t}, to obtain a plurality of differences Dⱼ=Mⱼ^{a}−Mⱼ^{t} and discarding all Dⱼ values lower than a predetermined threshold value to give a plurality of selected differences Dⱼ′;

(ix) comparing selected differences Dᵢ′ and Dⱼ′, preferably comprising overlapping theoretical fragments, and identifying those which are essentially identical; and optionally repeating steps (vi) to (ix), according to the number of different further digestion agents, obtaining selected differences Dₖ′/Dₗ′/Dₘ′, etc. The required mass of said mass altering moiety is thereby defined by said essentially identical Dᵢ′/Dⱼ′ values.

The method of the invention enables to characterize the type of the mass altering moiety, determining its mass, identity, as well as its location within the amino acid sequence, wherein the mass altering moiety may result from a chemical moiety added to the amino acid sequence either by post-translational modification that occurred in-vivo, or by modification that occurred in-vitro during sample preparation, or from change in the amino acid sequence (of one or more amino acids) caused by a mutation, alternative splicing, RNA editing, single nucleotide polymorphism (SNPs), a signal peptide cleavage, a difference in organism strain or species, or due to a database error.

The mass altering moiety being a chemical moiety added to the amino acid sequence, that can be characterized by the method of this invention, is selected from the group consisting of a sugar moiety, a lipidic moiety, an acyl moiety, an acidic moiety, biotin, a flavin, pyridoxal phosphate, and a moiety added by oxidation of sulphur in the peptide. When a mass altering moiety results from a post-translational modification, such modification can comprise acetylation, amidation, deamidation, farnesylation, formylation, geranylation, hydroxylation, methylation, myristoylation, phosphorylation, and sulphation.

When characterizing the assayed peptide according to the method of this invention, said peptide is first identified, and related to a database peptide, by any method comprising mass spectrometry, protein sequencing, immunoassay, chromatography, electrophoresis, protein chips, or antibody chips.

The digestion agents employed in the method of this invention can comprise either chemical agents or proteolytic enzymes, for example cyanogen bromide (CNBr), trypsin, chymotrypsin, Glu-C, Lys-C, AspN, elastase, or thermolysin.

In another aspect, this invention relates to a method for identifying a cleavage altering sequence which is present in an assayed peptide and is absent from a corresponding database peptide, or is present in a database peptide and is absent from an assayed peptide, wherein said cleavage altering sequence alters a cleavage site for at least one digestion agent used in the assay, the method comprising the steps of:

(i) treating the assayed peptide with a first digestion agent to obtain a first digestion product comprising a plurality of first assayed fragments; and determining the mass spectrum of the digestion product to obtain one or more mass values of the individual first assayed fragments Mᵢ^{a};

(ii) treating the assayed peptide with a further digestion agent to obtain a further digestion product comprising a plurality of further assayed fragments; and determining the mass spectrum of the further digestion product to obtain one or more mass values of the individual further assayed fragments Mⱼ^{a};

(iii) optionally repeating step (ii) according to the number of different further digestion agents, obtaining mass values of the individual further assayed fragments Mₖ^{a}, Mₗ^{a}, Mₘ^{a}, etc.;

(iv) optionally identifying the assayed peptide, in case of a peptide not identified earlier, by a suitable protein identification method;

(v) obtaining masses Mᵢ^{t} of the individual theoretical fragments of the database peptide corresponding to the assayed peptide, which fragments are obtained by the theoretical digestion of said database peptide with said first digestion agent;

(vi) obtaining masses Mⱼ^{t} of the individual theoretical fragments of the database peptide corresponding to the assayed peptide, which fragments are obtained by the theoretical digestion of said peptide with said further digestion agent;

(vii) optionally repeating step (vi) according to the number of different further digestion agents, obtaining masses Mₖ^{t}, Mₗ^{t}, Mₘ^{t}, etc., of the individual theoretical fragments.

(viii) comparing each of Mᵢ^{a} with each database value Mᵢ^{t}, to obtain a plurality of differences Dᵢ=Mᵢ^{a}−Mᵢ^{t}; discarding all Mia and Mi^{t} for which at least one of the Dᵢ values is lower than a predetermined threshold value; and thus identifying orphan Mᵢ^{a} that have no corresponding Mᵢ^{t}, and orphan Mᵢ^{t} that have no corresponding Mᵢ^{a};

(ix) comparing each of Mⱼ^{a} with each database value Mⱼ^{t}, to obtain a plurality of differences Dⱼ=Mⱼ^{a}−Mⱼ^{t}; discarding all Mja and Mⱼ^{t} for which at least one of the Dⱼ values is lower than a predetermined threshold value; and thus identifying orphan Mⱼ^{a} that have no corresponding Mⱼ^{t} and orphan Mj^{t} that have no corresponding Mⱼ^{a};

(x) optionally repeating step (ix) according to the number of different further digestion agents, and thus identifying orphan Mₖ^{a}, Mᵢ^{a}, Mₘ^{a}, etc., that have no corresponding Mₖ^{t}, Mₗ^{t}, Mₘ^{t}, etc., and identifying orphan Mₖ^{t}, Mₗ^{t}, Mₘ^{t}, etc that have no corresponding Mₖ^{a}, Mᵢ^{a}, Mₘ^{a}, etc.;

(xi) defining a first orphan region as the subset of the amino acid sequences of the database peptide which includes all the theoretical fragments corresponding to orphan Mᵢ^{t} for said first digestion agent; defining a further orphan region as the subset of the amino acid sequences of the database peptide which includes all the theoretical fragments corresponding to orphan Mⱼ^{t} for said further digestion agent; optionally repeating this for further digestion agents Mₖ^{t} etc.; and finally defining a peptide orphan region as the intersection of the first orphan region with all further orphan regions, thus consisting of a subset of sequences of the database peptide that were not identified by any of the digestion agents;

(xii) theoretically altering the amino acid sequence of said peptide orphan region, by adding, deleting or changing one or more amino acids thereof, to obtain altered database fragments; and calculating a set of theoretical values of masses Mₐₗₜ of said altered fragments;

(xiii) comparing each Mₐₗₜ with an orphan Mᵢ^{a}; orphan Mⱼ^{a}, orphan Mₖ^{a}, etc. and selecting those Mₐₗₜ of which the difference from an orphan Mⱼ^{a}, Mⱼ^{a}, Mₖ^{a} etc. is smaller than a predetermined threshold value. Mₐₗₜ representing the correct change will be selected based on a predetermined criterion, for example, confirmation by the largest number of different digestion agents; and thus identifying the amino acid sequence which is present only in the assayed peptide or in the database peptide as the altered database fragment contributing to said Mₐₗₜ.

The method of said another aspect of the invention enables to characterize the type of the mass of cleavage altering sequence, determining its identity, as well as its location within the amino acid sequence, wherein said cleavage altering sequence may result from a mutation, from single nucleotide polymorphism (SNPs), from RNA editing, from alternative splicing, from a signal peptide cleavage, from protein degradation, or from a database error, or wherein the difference between the assayed and the database peptide is caused by their being non-identical homologues, from a difference in organism strain or species.

When characterizing the assayed peptide according to this aspect of the invention, it is first identified, and related to a database peptide, by any method comprising mass spectrometry, protein sequencing, immunoassay, chromatography, electrophoresis, protein chips, or antibody chips.

The digestion agents employed in the method of this invention can comprise either chemical agents or proteolytic enzymes, including cyanogen bromide (CNBr), trypsin, chymotrypsin, Glu-C, Lys-C, AspN, elastase, or thermolysin.

This invention further relates to a kit for determining a mass altering moiety and/or cleavage altering sequence of a peptide, for use with mass spectroscopy, comprising two or more digestion agents, means for digesting peptides with the agents, and an instruction manual. In a preferred embodiment, this kit comprises at least two proteolytic enzymes.

### BRIEF DESCRIPTION OF THE DRAWINGS

The above and other characteristics and advantages of the invention will be better understood through the following illustrative and non-limitative examples, and with reference to the appended drawings, wherein:

FIG. 1 shows a scheme of the identification of a cleavage altering sequence;

FIG. 2 shows a scheme of the identification of a phosphorylation site and oxidized methionine;

FIG. 3 shows a scheme of the identification of a signal peptide cleavage site; and

FIG. 4 shows a scheme of the identification of an alternative splice variant.

### DETAILED DESCRIPTION OF THE INVENTION

The invention enables to identify mass altering moieties, which are present in the assayed peptide and not present in the database peptide, and vice versa. This includes moieties added by post-translational modification in-vivo, or by modifications that occur in-vitro during the sample preparation. Said mass altering moieties may also be the result of amino-acid alterations due to, for example, DNA polymorphism or mutation, in case the assayed peptide and the database peptide are from the same species/strain, or due to species/strain differences. Alternatively, they might result from difference of short stretch of amino acids (one or more amino acid residues) added, for example, through alternative splicing, or caused from species/strain differences.

By one aspect of this invention, the moiety is present in the database peptide but absent from the assayed peptide due to cleavage (for example of signal peptide), alternative splicing, species/strain differences, or any other difference between the protein form as it is in vivo and the form in which it is represented in the database.

When characterizing the assayed peptide according to the method of this invention, said peptide is first identified, and related to a database peptide, by any method comprising mass spectrometry, protein sequencing, immunoassay, chromatography, electrophoresis, protein chips, or antibody chips.

The present invention concerns a method for identifying a mass alteration moiety, on the condition that said moiety does not change cleavage sites of the assayed peptide as compared to the database peptide. Essentially the assayed peptide is treated with at least two digestion agents (for example two different proteolytic enzymes) to produce at least two digestion products, each having a plurality of individual assayed fragments. Then, the digestion products are subjected to mass spectrometry analysis to give mass spectra with individual peaks representing the plurality of fragment masses. The masses of the actual fragments obtained by the two digestion agents are then compared with the theoretical masses of fragments which would have been obtained by treatment of the corresponding database peptide with the respective two different digestion agents. This means that each mass produced by actual digestion of the assayed peptide is compared with all the masses of the fragments obtained by theoretical digestion of the database peptide with the same digestion agent.

Masses of some of the assayed fragments are identical with the masses of the theoretical fragments of the database peptide (obtained by digestion with the same digestion agent), the identity being recognized when the difference between assayed and theoretical fragments is lower than a certain threshold value. Said threshold value, which is the minimal mass difference that is significant, is predetermined according to the accuracy of the mass spectrometric experiment, taking into account experimental errors of all the methods and equipment used. Said threshold value is preferably predetermined as a value from 0.05 to 0.5 Dalton, still more preferably from 0.1 to 0.2 Dalton. This value can be, for example 0.2 Dalton.

“Real” differences, i.e. differences greater than the threshold value, between masses of the assayed fragments and the database peptide fragments are selected, in respect of each of the digestion agents, separately. These selected differences in mass may be due to a plurality of reasons, the most obvious one being that two completely different fragments, from different regions of the assayed peptide and the database peptide, were used to calculate the difference (as indicated all masses are compared to each other); differences may also be due, for example, to “contamination” of the mixture by non-assayed peptides.

However, the differences which are to be identified by the method of the invention are real mass differences caused by the presence or absence of said mass altering moiety. If the difference between an assayed fragment and a database peptide fragment, both “treated” by the same first digestion agent, is identical to the difference of an assayed fragment and a database peptide fragment, both “treated” by the further digestion agent, and if, furthermore, the first database peptide fragment and the further database peptide fragment overlap, i.e., have a common subsequence, then this is a strong indication that this mass difference is due to the presence or absence of a real physical moiety, and that the difference is equal to the mass of said mass altering moiety. The difference can have a positive value (for example where a methionine has been oxidized, adding 16 Daltons to the fragment) or a negative value (for example where cysteine has changed to dehydroalanine, subtracting 34 Daltons).

It is important to stress that peptides and their differences are compared only when these theoretical peptides overlap. Thus, if the protein has been digested with both trypsin and Glu-C, the analysis will take into account theoretical tryptic peptides with theoretical Glu-C peptides that overlap, and test the peak list to see if there are any differences that indicate a mass altering moiety on the overlapping part of the amino acid sequences of the two peptides. It would be appreciated by anyone skilled in the art that the more accurate the mass spectrometry instrument, the easier it would be to recognize the true essentially identical differences from the false ones. Thus, if we assume 100 mass values (peaks) that were obtained from the instrument; and 100 theoretical values from the database peptide, than we can expect 10,000 differences, most of which will lie in the region between −3000 and +3000 Daltons. Statistically, a number of these differences will coincide with the experiment made with a further digestion agent. Evidently, the better the accuracy of the machine, the smaller is the chance of obtaining such coincidences (“false positives”). It is easy to see that doubling the accuracy will halve the number of such false positives.

In order to decrease the number of these false positives, it is helpful to calibrate the machine's results as accurately as possible. We herein disclose a method by which this may be carried out. Since we already have at this stage a list of theoretical fragment masses together with their corresponding assayed masses, it is easy to use these data pairs for calibration. For example, a linear fit can be used, or a polynomial fit or any other suitable numerical method. This calibration can help to increase the machine's accuracy and decrease the number of false positives.

By knowing the mass of the mass altering moiety and its location, it is possible to hypothesize on the nature of said moiety, as shown, for example, by Wilkins M. R. et al. [J.Mol.Biol. 289 (1999) 645-657] who, however, used only a single digestion agent. For example, a mass altering sequence of 79.7 on a sequence of amino acids GSW is a strong hint that serine (S) was phosphorylated.

Thus, the present invention concerns a method for determining a mass of a mass altering moiety, which is present either in an assayed peptide or in a corresponding database peptide, the method comprising: (i) treating the assayed peptide with a first digestion agent to obtain a first digestion product comprising a plurality of first assayed fragments; and determining the mass spectrum of the digestion product to obtain one or more mass values of the individual first assayed fragments Mᵢ^{a}; (ii) treating the assayed peptide with a further digestion agent to obtain a further digestion product comprising a plurality of further assayed fragments; and determining the mass spectrum of the further digestion product to obtain one or more mass values of the individual further assayed fragments Mⱼ^{a}; (iii) optionally repeating step (ii) according to the number of different further digestion agents, obtaining mass values of the individual further assayed fragments Mₖ^{a}, Mₗ^{a}, Mₘ^{a}, etc.; (iv) optionally identifying the assayed peptide, in case of a peptide not identified earlier, by a suitable protein identification method;(v) obtaining masses Mᵢ^{t} of the individual theoretical fragments of the database peptide corresponding to the assayed peptide, which fragments are obtained by the theoretical digestion of said database peptide with said first digestion agent; (vi) obtaining masses Mⱼ^{t} of the individual theoretical fragments of the database peptide corresponding to the assayed peptide, which fragments are obtained by the theoretical digestion of said database peptide with said further digestion agent; (vii) comparing each of Mᵢ^{a} with each database value Mᵢ^{t}, to obtain a plurality of differences Dᵢ=Mᵢ^{a}−Mᵢ^{t} and discarding all Dᵢ values lower than a predetermined threshold value to give a plurality of selected differences Dᵢ′; (viii) comparing each of Mⱼ^{a} with each database value Mⱼ^{t}, to obtain a plurality of differences Dⱼ=Mⱼ^{a}−Mⱼ^{t} and discarding all Dⱼ values lower than a predetermined threshold value to give a plurality of selected differences Dⱼ′; (ix) comparing selected differences Dᵢ′ and Dⱼ′, preferably comprising overlapping theoretical fragments, and identifying those which are essentially identical; and optionally repeating steps (vi) to (ix), according to the number of different further digestion agents, obtaining selected differences Dₖ′/Dᵢ′/Dₘ′, etc. The required mass of said mass altering moiety is thereby defined by said essentially identical Dᵢ′/Dⱼ′ values.

The above description represents a procedure that can serve in computerization and automation of the method according to the invention.

The digestion agent can be a chemical agent or a proteolytic enzyme. Examples of such agents, without being limited to them, are cyanogen bromide, trypsin, chymotrypsin, Glu-C, Lys-C, AspN, elastase, and thermolysin. It should be appreciated that the “further digestion agent” may be a second digestion agent (different from the first digestion agent), a mixture of two or more digestion agents (including the first agent) but may also be a third, fourth and fifth digestion agent. In such a case step (ii) is repeated for a number of times corresponding to the number of different further digestion agents; step (vi) is repeated the number of times of all the further digestion agents, and in step (ix) all the selected differences are compared to each other in order to identify those which are essentially the same. The greater the number of digestion agents is, the greater is the confidence and the accuracy in the results of the present invention, enabling better approximation to the real masses and improved localization of a modification on the protein sequence.

The method of the invention enables to determine where (i.e. in which amino acid region) the mass altering moiety is present. If, for example, the moiety is a post-translational modification being an addition of a sugar moiety, it is possible to know in which short stretch of amino acid this moiety is present. The D_{i′} D_{j′} values, which were substantially the same, were obtained by the difference between specific Mᵢ^{a}−Mᵢ^{t} and Mⱼ^{a}−Mⱼ^{t}, respectively. The knowledge of amino acid sequence of the said theoretical fragments Mᵢ^{t} and Mⱼ^{t} enables to deduce the amino acid sequence of the overlapping region of these two fragments. This said amino acid sequence is the sequence comprising the mass altering moiety. The more digestion agents are used, the smaller the overlapping sequence common to all the selected fragments is , which enables to pin-point more precisely the region of the addition moiety.

By another aspect, the present invention relates to a method for the identification of a cleavage altering sequence, i.e. an amino acid sequence which is present in an assayed peptide and absent from the corresponding database peptide, or vice versa, wherein due to the deletion or addition of the amino acid sequence in the assayed peptide a new cleavage site was created or a previous cleavage site was deleted. This means that digesting the assayed peptide with the same digestion agents applied theoretically for the digestion of the database peptide may cause production of one or more completely novel fragments having novel amino acid sequences. This method may be used, for example, to identify a signal peptide that was cleaved off a protein, an amino acid change which effects the cleavage pattern, or an alternative splice variant form of a protein which is not present in the database.

The assayed peptide is treated with digestion agents and the masses of the fragments obtained by said digestions are again correlated with the theoretical masses of the corresponding theoretical fragments obtained from a database peptide “treated” theoretically with the same digestion agents. Fragments present in the mass spectra of a digestion product which have no corresponding theoretical masses of the individual fragments of the corresponding database peptide, and fragments of the database peptide obtained by “theoretical digestion” with a digestion agent which do not have corresponding masses of the assayed peptide, i.e. orphan fragments, are found. Orphan Mᵢ^{t}, Mⱼ^{t}, Mₖ^{t}, etc and orphan Mᵢ^{a}, Mⱼ^{a}, Mₖ^{a}, etc are used for finding the peptide orphan region and identifying the cleavage altering sequence.

Thus the sequence of the database peptide, which is suspected as having been changed, is mapped by marking all fragments that have so far been detected with any of the cleaving agents that were used. Where a cleavage altering sequence has taken place in the assayed peptide, a gap will become evident on the original database peptide map. Thus, when a signal peptide has been cleaved, the entire N-terminus of the original database peptide will have been left unmarked. This is the area that is suspect of having had its cleavage pattern changed, and the place where the analysis of the orphan masses will take place.

This analysis is done by obtaining a first orphan region and a further orphan region, i.e. subsets of the amino acid sequences of the database peptide which include all fragments corresponding to orphan Mᵢ^{t} and Mⱼ^{t}, respectively, followed by obtaining the peptide orphan region, which is the intersection of the first orphan region with the further orphan region, and eventually with all other further orphan regions, thus consisting of a subset of the sequences of the peptide protein that were not identified by any of the digestion agents. It means that the peptides corresponding to orphan masses are mapped on the protein, and only those regions which consistently do not match the assayed masses (considering all the digestion products analyzed), are considered as potentially “missing” from the assayed protein. Then, the database sequence of this peptide orphan region is trimmed, one amino acid at a time, until a confirmation is obtained, based on a pre-determined criterion. This criterion may be, for example, confirmation by the largest number of different digestion agents, i.e. if one change was confirmed by two different digestion agents, and another change was confirmed by three digestion agents, the latter will be considered as the correct change.

For example, in a signal peptide cleavage, let us suppose that none of the 20 extreme acids at the N-terminus has been observed by the analysis. In this case, the analysis will repetitively remove the extreme N-terminal amino acid from the peptide orphan region, and test whether masses of the resulting theoretical peptides match orphan Mᵢ^{a}, Mⱼ^{a}, etc. The correct cleavage point will be selected based on said pre-determined criterion.

Of course due to statistical reasons, it is much easier to delete theoretically amino acids from the database peptide, than to add or change amino acids thereto since all 20 naturally occurring amino acids have to be tried. However, sometimes, relevant genomic information exists, either in the form of the corresponding RNA sequence, or the corresponding DNA sequence. In such cases, one can use the genomic information to restrict the number of possible amino acid sequence changes to be examined.

Thus, the present invention concerns a method for identifying a cleavage altering sequence, comprising: (i) treating the assayed peptide with a first digestion agent to obtain a first digestion product comprising a plurality of first assayed fragments; and determining the mass spectrum of the digestion product to obtain one or more mass values of the individual first assayed fragments Mᵢ^{a}; (ii) treating the assayed peptide with a further digestion agent to obtain a further digestion product comprising a plurality of further assayed fragments; and determining the mass spectrum of the further digestion product to obtain one or more mass values of the individual further assayed fragments Mⱼ^{a}; (iii) optionally repeating step (ii) according to the number of different further digestion agents, obtaining mass values of the individual further assayed fragments Mₖ^{a}, Mₗ^{a}, Mₘ^{a}, etc.; (iv) optionally identifying the assayed peptide, in case of a peptide not identified earlier, by a suitable protein identification method; (v) obtaining masses Mᵢ^{t} of the individual theoretical fragments of the database peptide corresponding to the assayed peptide, which fragments are obtained by the theoretical digestion of said database peptide with said first digestion agent; (vi) obtaining masses Mⱼ^{t} of the individual theoretical fragments of the database peptide corresponding to the assayed peptide, which fragments are obtained by the theoretical digestion of said peptide with said further digestion agent; (vii) optionally repeating step (vi) according to the number of different further digestion agents, obtaining masses Mₖ^{t}, Mᵢ^{t}, Mₘ^{t}, etc., of the individual theoretical fragments; (viii) comparing each of Mᵢ^{a} with each database value Mᵢ^{t}, to obtain a plurality of differences Dᵢ=Mᵢ^{a}−Mᵢ^{t}; discarding all Mᵢ^{a} and Mᵢ^{t} for which at least one of the Dᵢ values is lower than a predetermined threshold value; and thus identifying orphan Mᵢ^{a} that have no corresponding Mᵢ^{t}, and orphan Mᵢ^{t} that have no corresponding Mᵢ^{a}; (ix) comparing each of Mⱼ^{a} with each database value Mⱼ^{t}, to obtain a plurality of differences Dⱼ=Mⱼ^{a}−Mⱼ^{t}; discarding all Mⱼ^{a} and Mⱼ^{t} for which at least one of the Dⱼ values is lower than a predetermined threshold value; and thus identifying orphan Mⱼ^{a} that have no corresponding Mⱼ^{t}, and orphan Mⱼ^{t} that have no corresponding Mⱼ^{a}; (x) optionally repeating step (ix) according to the number of different further digestion agents, and thus identifying orphan Mₖ^{a}, Mₗ^{a}, Mₘ^{a}, etc., that have no corresponding Mₖ^{t}, Mₗ^{t}, Mₘ^{t}, etc., and identifying orphan Mₖ^{t}, Mₗ^{t}, Mₘ^{t}, etc that have no corresponding Mₖ^{a}, Mₗ^{a}, Mₘ^{a}, etc.; (xi) defining a first orphan region as the subset of the amino acid sequences of the database peptide which includes all the theoretical fragments corresponding to orphan Mᵢ^{t} for said first digestion agent; defining a further orphan region as the subset of the amino acid sequences of the database peptide which includes all the theoretical fragments corresponding to orphan Mⱼ^{t} for said further digestion agent; optionally repeating this for further digestion agents Mₖ^{t} etc.; and finally defining a peptide orphan region as the intersection of the first orphan region with all further orphan regions, thus consisting of a subset of sequences of the database peptide that were not identified by any of the digestion agents; (xii) theoretically altering the amino acid sequence of said peptide orphan region, by adding, deleting or changing one or more amino acids thereof, to obtain altered database fragments; and calculating a set of theoretical values of masses Mₐₗₜ of said altered fragments; (xiii) comparing each Mₐₗₜ with an orphan Mᵢ^{a}; orphan Mⱼ^{a}, orphan Mₖ^{a}, etc. and selecting those Mₐₗₜ of which the difference from an orphan Mᵢ^{a}, Mⱼ^{a}, Mₖ^{a} etc. is smaller than a predetermined threshold value. Mₐₗₜ representing the correct change will be selected based on a pre-determined criterion, for example, confirmation by the largest number of different digestion agents; and thus identifying the amino acid sequence which is present only in the assayed peptide or in the database peptide as the altered database fragment contributing to said Mₐₗₜ.

The above description represents a procedure that can serve in computerization and automation of the method according to the invention.

The digestion agent can be a chemical agent or a proteolytic enzyme. Examples of such agents, without being limited to them, are cyanogen bromide, trypsin, chymotrypsin, Glu-C, Lys-C, AspN, elastase, and thermolysin. It should be appreciated that the “further digestion agent” may be a second digestion agent (different from the first digestion agent), a mixture of two or more digestion agents (including the first agent), but may also be a third, fourth and fifth digestion agent. In such a case, steps (ii), (vi) and (ix) are repeated the number of times corresponding to the number of different further digestion agents. Again, the greater the number of digestion agents is, the greater is the confidence and the accuracy in the results of the present invention.

The invention also relates to a kit for determining a mass altering moiety and/or cleavage altering sequence of a peptide for use with mass spectroscopy, comprising two or more digestion agents, means for digesting peptides with the agents, and an instruction manual. The kit comprises preferably at least two proteolytic enzymes. The part of the kit can be buffers, salt solutions, enzyme solutions or ampules with lyophilized enzymes, etc. The kit can comprise glass or plastic equipment for portioning the solutions, mixing the reagents, and preparing samples for MS.

### EXAMPLE 1

### Identification of a Post-Translational Modification

The assayed protein is identified by the standard methods (for example as given in the references above, Wise et al.; Jonscher et al.; Yates et al.; Gevaret et al.; and Perkins et al.) as corresponding to database peptides known as COQ7^{—}Human having the following sequence:

KMWDQEKDHLKKFNELMVMFRVRPTVLMPLWNVLGFALGAGTALLG

The first digestion agent is trypsin, which selectively cleaves at the C-terminus of R and K, not before P. Thus, theoretical digestion produces 6 fragments as follows:

1 2 3 4 5
K; MWDQEK; DHLK; K; FNELMVMER;

6
VRPTVLMPLWNVLGFALGAGTALLG

The masses of the theoretical database fragments (defined as Mᵢ^{t}) are Mᵢ^{t}1, Mᵢ^{t}2, Mⱼ^{t}3, Mⱼ^{t}4, Mᵢ^{t}5, and M6, respectively.

The second digestion agent is chymotrypsin. As chymotrypisin selectively cleaves at the C-terminus of F, Y, W, the following fragments were expected:

1 2 3 4 5
KMW; DQEKDHLKKF; NELMVMF; RVRPTVLMPLW; NVLGF;

6
ALGAGTALLG

The masses of the theoretical fragments (defined as Mⱼ^{t}) are Mⱼ^{t}1, Mⱼ^{t}2, Mⱼ^{t}3, Mⱼ^{t}4, Mⱼ^{t}5, and Mⱼ^{t}6, respectively.

The assayed peptide is modified by a post-translational modification to have modification X attached thereto as follows:

```
    X
    |
KMWDQEKDHLKKFNELMVMFRVRPTVLMPLWNVLGFALGAGTALLG
```

The actual assayed peptide is treated in one reaction vessel with trypsin and in the other reaction vessel with chymotrypsin. Both digests are analyzed by mass spectrometry. The mass spectrum of the trypsin digest shows: Mᵢ^{a}3, Mᵢ^{a}4, Mᵢ^{a}6 and Mᵢu (u standing for unknown). As is known to one skilled in the art, not all theoretical fragments are experimentally observed.

The masses Mᵢ^{a}3, Mᵢ^{a}₄ and Mᵢ^{a}6 of the assayed peptide are substantially identical (i.e. the difference is below a threshold value) to Mᵢ^{t}3; Mᵢ^{t}4 and Mᵢ^{t}6 of the database peptide. However, Mᵢ^{a} u has a mass that does not correspond to any of the fragments of the database peptide.

The mass spectrum of the chymotrypsin digest shows: Mⱼ^{a}2; Mⱼ^{a}4, Mⱼ^{a}6 and Mⱼ^{a}u, the first three being substantially identical to the corresponding fragments Mⱼ^{t}2′ Mⱼ^{t}4 and Mⱼ^{t}6.

Then, the two unknown masses Mᵢ^{a}u and Mⱼ^{a}u obtained by treatment with trypsin and chymotrypsin, respectively, are compared with each of the fragments, of the database peptide. This means that Mᵢ^{a}u is compared with Mᵢ^{t}1-6 masses of the database peptide theoretically treated with trypsin, whereas Mⱼ^{a}u is compared with Mⱼ^{t}1-6 of the database peptide theoretically treated with chymotrypsin.

It is found that: Mᵢ^{a}u−M^{t}ᵢ2=Mⱼ^{a} u−Mⱼ^{t}1=X. Thus, X is considered to be significant, that is the mass of an altering m As is clear to one skilled in theart, the mass of the moiety gives a strong indication as to the exact nature of the moiety.

Since Mᵢ^{t}2 and Mⱼ^{t}1 each contributed to this identical difference giving the mass X, it is possible to determine the sequence of the fragment which has a mass Mᵢ^{t}2 (MWDQEK), and the sequence of fragment which has a mass Mⱼ^{t}1 (KMW). The region which overlaps in these two sequences is: MW, and this is the sequence where the modification X is present.

If more than two proteases are used, it may be possible to pin-point the modification point more precisely.

### EXAMPLE 2

### Identification of a Cleavage Altering Sequence

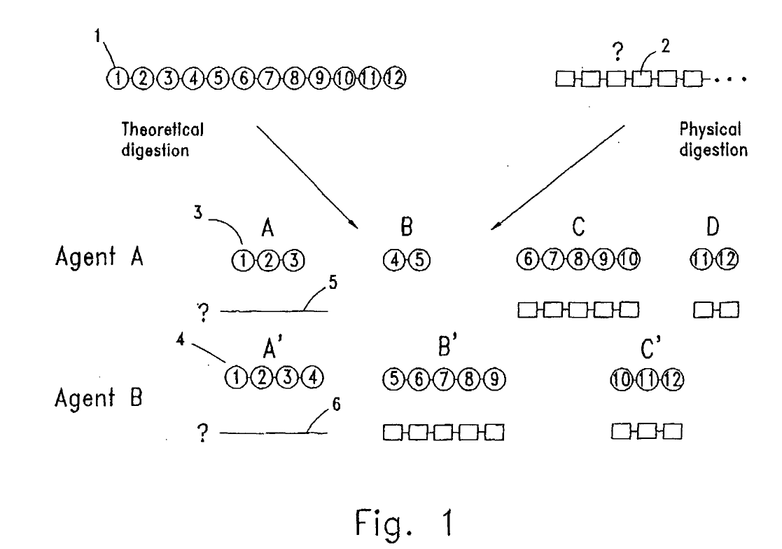

Reference is made to FIG. 1, which shows schematically the method of the invention for identification of a cleavage altering sequence. First, by methods known in the art (after “digestion” with identical agents), a database peptide is identified which corresponds to the assayed peptide. The database peptide (1) is composed of 12 amino acids shown schematically as numbers. The corresponding assayed peptide (2) has an unknown sequence.

Theoretical digestion of the database peptide (1) with digestion agent A results in 4 fragments: A, B, C, D (3) and theoretical digestion of the database with digestion agent B results in 3 fragments A′, B′, C′ (4). The assayed peptide (2) is physically digested, in two separate reaction vessels, with agents A and B.

Comparison to the masses of fragments of assayed peptide digested by agent A shows masses of fragments corresponding to C, D of the database peptide and another assayed peptide having an orphan mass (5) (i.e. having no database counterpart). Database fragments A and B have no counterpart in assayed fragment masses.

Comparing to the masses of fragments of assayed peptide digested by agent B shows fragments corresponding to B′ and C′ and another assayed peptide orphan mass (6). Fragment A′ have no counterpart mass in the assayed fragments.

Now fragment A′ is altered by sequentially eliminating each of the amino acids no. 1, 2, 3 or 4 and comparing the masses of the theoretically altered fragments with orphan mass (6). Only deletion of amino acid no. 4 results in a mass identical with that of orphan (6). To validate this result, digestion with agent A is considered, and it is found that orphan mass (5) is higher than each of the mass of any of database fragments A and B which have no counterpart. Thus it could be assumed that this orphan mass (5) is a result of the combination masses of A and B due to loss of a cleavage site resulting in one longer peptide. This could be due to loss of amino acid no. 3 or 4. Calculation of the mass of the theoretical alterations shows that only loss of amino acid no. 4 and combination of fragments A and B results in orphan mass (5) validating the results obtained with digestion agent B.

### Experimental—Materials and Methods

### In Gel Digestion

The procedure was performed at room temperature, unless specified otherwise. 1 μg of casein alpha (Sigma-Aldrich, St. Louis, Mo., USA) was separated on 1D-SDS-15%PAGE. Protein bands were excised from the gel, washed twice with water and chopped. The gel pieces were incubated for 10-15 min with acetonitrile. This incubation leads to shrinking of the gel pieces, which were then dried for 5 min in a vacuum centrifuge. The dried gel pieces were reswollen with reduction buffer (100 mM NH₄HCO₃, 10 mM dithiothreitol (DTT)), and incubated at 56° C. for 30 min. The reduction buffer was then replaced with acetonitrile for 10-15 min incubation. The shrinked gel pieces were dried for 5 min in a vacuum centrifuge and reswollen with alkylation buffer (55 mM iodoacetamide and 100 mM NH₄HCO₃) for 20 min in the dark. The alkylation buffer was replaced with 100 mM NH₄HCO₃ for 15 min incubation, which was then replaced with acetonitrile for 10-15 min incubation. The shrinked gel pieces were dried for 5min in a vacuum centrifuge and reswollen with just enough of protease solution to cover the rehydrated gel. The protease solution contained 12.5 ng/μl trypsin or Lys-C or Glu-C or chymotrypsin in digestion buffer (50 mM NH₄HCO₃ and 5 mM CaCl₂). After 30 min incubation at 4° C., protease solution that was not absorbed in the gel was discarded, and digestion buffer was added just enough to cover the gel pieces. Digestion was performed at 37° C. for 16 h, after which the supernatant was transferred to a new tube. Then, peptides were extracted from the gel matrix by the following steps: (i) 10 min incubation with 10-15 μl of 25 mM NH₄HCO₃, the supernatant was recovered, (ii) 15 min incubation at 37° C. with acetonitrile (1-2 times the volume of the gel particles), the supernatant was recovered, (iii) 15 min incubation at 37° C. with 40-50 μl of 5% formic acid, the supernatant was recovered, and (iv) 15 min incubation at 37° C. with acetonitrile (1-2 times the volume of the gel particles), the supernatant was recovered. The supernatants from all the above extraction steps (i-iv) were pooled and joined with the supernatant from the digestion reaction. The sample was dried down in a vacuum centrifuge, and recovered with 5 μl 0.1% TFA of which 0.5 μl was taken to the mass spectrometry analysis.

### Sample Preparation for MALDI-TOF Mass Spectrometer Analysis

Samples were prepared on the MALDI plate by the following steps: (i) saturated matrix solution of α-cyano-4-hydroxycinnamic acid (Sigma-Aldrich, St. Louis, Mo., USA) in ethanol was applied on the target area, and left to dry, (ii) 0.5 μl from the in-gel digestion reaction was mixed with 0.5 μl saturated matrix solution of α-cyano-4-hydroxycinnamic acid in 0.1%TFA/acetonitrile (1 :1 v/v), (iii) the mixed sample was then spotted on top of the dried first layer, and left to dry, (iv) the dried samples were washed with 5 μl of 0.1%TFA.

### MALDI Analysis

Samples were analyzed using REFLEX III matrix-assisted laser desorption/ionization (MALDI) time-of-flight (TOF) mass spectrometer (Bruker Daltonik, Bremen, Germany) equipped with a 337 nm nitrogen laser. Mass spectra were acquired in the positive reflectron mode. Each spectrum was a sum of 200 individual laser pulses. One-point mass calibration was performed using an internal standard (matrix peak m/z 1060.1)

### EXAMPLE 3

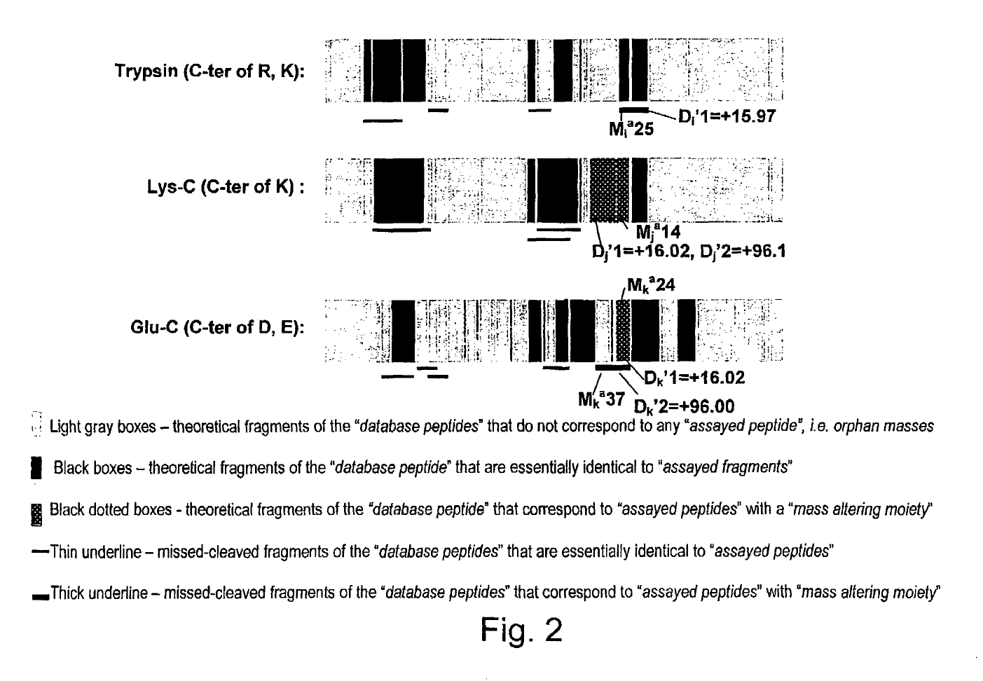

### Identification of phosphorylation Site and oxidized methionine (FIG. 2)

(i) The assayed protein Casein alpha was digested by the standard methods (described in the Materials and Methods section) with three digestion agents: trypsin, Lys-C, and Glu-C.

(ii) The mass spectrum of each of the three digestion products comprising of plurality of “assayed fragments”, was analyzed to obtain the mass values of the individual “assayed fragments” (Mᵢ^{a}; Mⱼ^{a}; and Mₖ^{a}, respectively).

(iii) The masses Mᵢ^{t}, Mⱼ^{t} and Mₖ^{t} of the individual theoretical fragments of the database peptide (Casein alpha—CAS1_BOVIN in SwissProt database) corresponding to the assayed peptide, which fragments are obtained by the theoretical digestion of said database peptide with said digestion agents, were obtained;(iv) Each Mᵢ^{a}; Mⱼ^{a}; and Mₖ^{a} were compared with the fragment masses of the theoretically digested database peptide, Mᵢ^{t}; Mⱼ^{t}; and Mₖ^{t}, respectively. A plurality of differences was obtained for each digestion product (Dᵢ=Mᵢ^{a}−Mᵢ^{t}; Dⱼ=Mⱼ^{a}−Mⱼ^{t}; Dₖ=Mₖ^{a}−Mₖ^{t}). Theoretical fragments that are essentially identical to assayed fragments (i.e. Mᵢ^{t}, Mⱼ^{t} and Mₖ^{t} for which at least one of the Dᵢ, Dⱼ and Dₖ values, respectively, is lower than 0.11 Da) are marked as black boxes in FIG. 2.

(v) Dᵢ; Dⱼ; and Dₖ which had a value lower than 0.11 Da were discarded, to select Dᵢ′; Dⱼ′; and Dₖ′.

(vi) Dᵢ′; Dⱼ′; and Dₖ′ resulting from overlapping theoretical fragments were compared to each other. As shown in FIG. 2:

a. Dᵢ′1, Dⱼ′1 and Dₖ′1 are substantially the same (16±0.03 Da). The amino-acid sequences of the relevant database fragments Mᵢ^{t}; Mⱼ^{t} and Mₖ^{t} (denoted in FIG. 2 as Mᵢ^{t} 25; Mⱼ^{t} 14 and Mₖ^{t}24) are: LHSMKEGIHAQQK; VPQLEIVPNSAEERLHSMK; and RLHSMKE. The overlapping sequence between the above fragments is LHSMK. A possible mass altering moiety having a mass of 15.99 Da is oxidation of methionine (M), which is included in the overlapping sequence.

b. Dⱼ′2 and Dₖ′2 are substantially the same (96±0.1 Da). The amino-acid sequences of the relevant database fragments Mⱼ^{t} and Mₖ^{t} (denoted in FIG. 2 as Mⱼ^{t}14 and Mₖ^{t}37) are: VPQLEIVPNSAEERLHSMK; and IVPNSAEERLHSMKE. The overlapping sequence of the above fragments is IVPNSAEERLHSMK. A possible mass altering moiety having a mass of 95.95 Da is the combination of phosphorylation (+79.96 Da) of serine (S) and oxidation (+15.99 Da) of methionine (M) (the same residue as in the above example).

### EXAMPLE 4

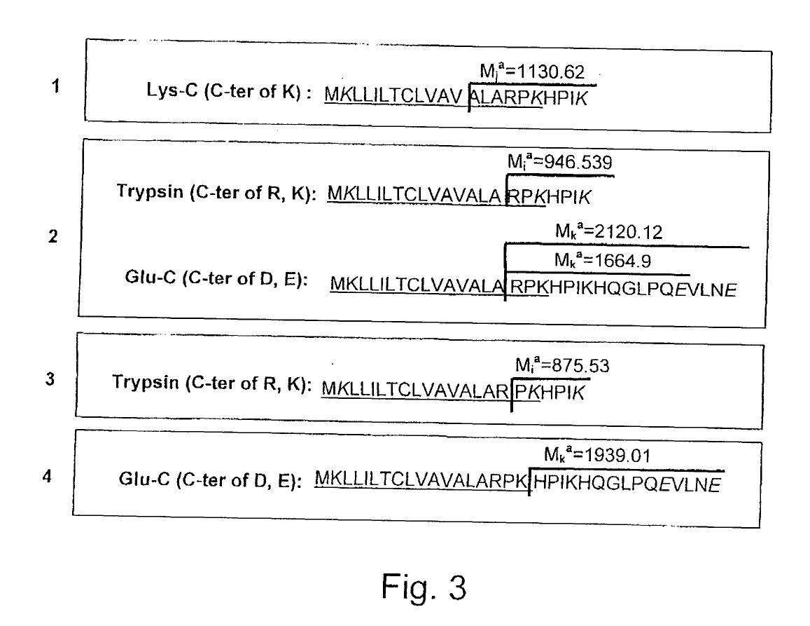

### Identification of Signal Peptide Cleavage Site (FIG. 2 and FIG. 3)

The assayed protein Casein alpha was digested and the digestion products were analyzed as in steps (i)-(iv) of Example 3. The following procedure was applied in order to identify the cleavage site of a possible signal peptide:

a. All Mᵢ^{a}; Mⱼ^{a}, Mₖ^{a} and Mᵢ^{t}, Mⱼ^{t}, Mₖ^{t} for which at least one of the Dᵢ, Dⱼ, Dₖ values, respectively, was lower than 0.11 Da, were discarded; and thus orphan Mᵢ^{a}; Mᵢ^{a}, Mₖ^{a} that have no corresponding Mᵢ^{t}, Mⱼ^{t}, Mₖ^{t}, respectively, and orphan Mᵢ^{t}, Mⱼ^{t}, Mₖ^{t} that have no corresponding Mᵢ^{a}; Mⱼ^{a}, Mₖ^{a}, respectively, were identified.

b. First and further orphan regions were defined at the N-terminus of the database peptide (CAS1_BOVIN). The regions comprising amino acids 1-18, 1-23 and 1-29, were defined as orphan regions, considering the digestion products of trypsin, Lys-C and Glu-C, respectively. Thus, the peptide orphan region (i.e. intersection of the first orphan region with all further orphan regions) comprises amino acids 1-18.

c. One amino acid was theoretically removed from the N-terminus of the peptide orphan region. The theoretical altered masses of the resulting peptides of the different proteases, trypsin, Lys-C, and Glu-C (Mₐₗₜ) were calculated, and searched in the mass lists of the orphan assayed fragments (orphan Mᵢ^{a}; Mⱼ^{a}; and Mₖ^{a}, respectively).

d. Step c was repeated until amino acid 18 (the C-terminus of the peptide orphan region).

e. Cleavage points supported by several orphan assayed fragments (orphan Mᵢ^{a}; Mⱼ^{a}; and Mₖ^{a}) deriving from different digestion agents were searched. Masses of orphan assayed fragments (orphan Mᵢ^{a}; Mⱼ^{a}; and Mₖ^{a}) that were matched to theoretically calculated peptides during the above procedure are shown in FIG. 3 (italic letters denote the protease cleavage site, underlined letters denote amino acids comprising the peptide orphan region). In box 1,3 and 4, in which the theoretical cleavage occurred C-terminal to V-12, R-16 and K-18, respectively, only one “assayed fragment” was matched. This may result from contamination. However, in box 2, in which the theoretical cleavage occurred C-terminal to A- 15, three different assayed fragments derived from the proteolysis by two different proteases (trypsin and Glu-C), were matched. Thus, this cleavage site is considered as the real cleavage site.

### EXAMPLE 5

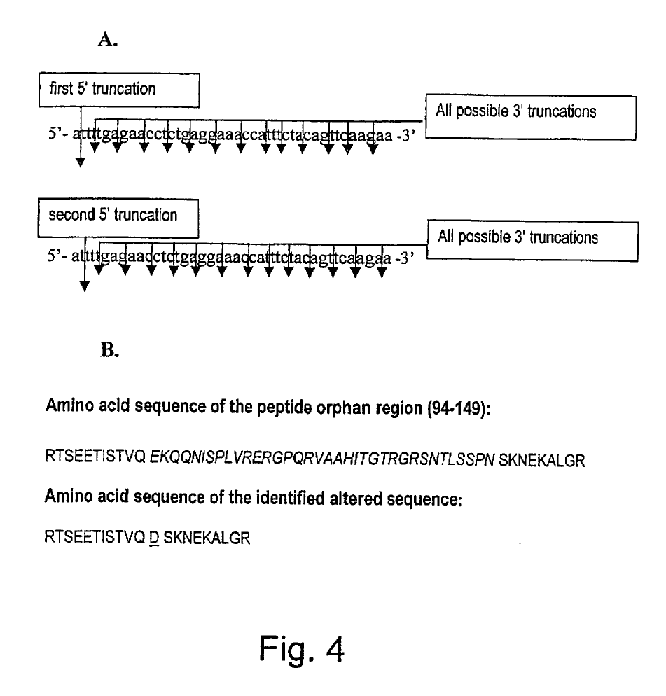

### Identification of an Alternative Splice Variant (FIG. 4)

(i) The assayed protein was digested by the standard methods (described in the Methods section) with three digestion agents: trypsin, Lys-C, and chymotrypsin.

(ii) The mass spectrum of each of the three digestion products, comprising plurality of “assayed fragments”, was analyzed to obtain the mass values of the individual “assayed fragments” (Mᵢ^{a}; Mⱼ^{a}; and Mₖ^{a}, respectively).

(iii) A protein database (containing proteins from SwissProt) was searched to find the corresponding “database peptide” that matches the PMF of Mᵢ; Mⱼ; and Mₖ. The protein TN10_HUMAN was identified.

(iv) The masses Mᵢ^{t}, Mⱼ^{t} and Mₖ^{t} of the individual theoretical fragments of the said database peptide corresponding to the assayed peptide, which fragments are obtained by the theoretical digestion of said database peptide with said digestion agents, were obtained;

(v) Each Mᵢ^{a}; Mⱼ^{a}; and Mₖ^{a} were compared with the fragment masses of the theoretically digested database peptide, Mᵢ^{t}; Mⱼ^{t}; and Mₖ^{t}, respectively. A plurality of differences was obtained for each digestion product (Dᵢ=Mᵢ^{a}−Mᵢ^{t}; Dⱼ=Mⱼ^{a}−Mⱼ^{t}; Dₖ=Mₖ^{a}−Mₖ^{t}).

(vi) All Mᵢ^{a}; Mⱼ^{a}, Mₖ^{a} and Mᵢ^{t}, Mⱼ^{t}, Mₖ^{t} for which at least one of the Dᵢ, Dⱼ, Dₖ values, respectively, was lower than 0.035 Dalton, 50 ppm and 70 ppm, respectively, were discarded; and thus orphan Mᵢ^{a}; Mⱼ^{a}, Mₖ^{a} that have no corresponding Mᵢ^{t}, Mⱼ^{t}, Mₖ^{t}, respectively, and orphan Mᵢ^{t}, Mⱼ^{t}, Mₖ^{t} that have no corresponding Mᵢ^{a}; Mⱼ^{a}, Mₖ^{a}, respectively, were identified.

(vii) First and further orphan regions were defined. The region comprising amino acids 1-149, 91-143 and 94-155, were defined as orphan regions, considering the digestion products of trypsin, Lys-C and chymotrypsin, respectively Thus, a peptide orphan region (i.e. intersection of the first orphan region with all further orphan regions) comprising amino acids 94-149, was defined.

(viii) The nucleotide sequence of the RNA transcript coding for TN10_HUMAN was obtained from the GenBank (gi1149557). The nucleotides coding for the peptide orphan region were mapped (367-534nt).

(ix) The said nucleotide sequence coding for the peptide orphan region was truncated by one nucleotide from its 5′end, and then the sequence was truncated from its 3′end in all possible sites, so that the total number of nucleotides between the 5′ and 3′ truncations will be an integer times 3, so as to keep the reading frame (the two initial steps are illustrated in FIG. 4A).

(x) Step (ix) was repeated successively.

(xi) All the truncated nucleotide sequences resulting from steps (ix) and (x) were translated into amino acid sequences, and the theoretical altered masses of the resulting peptides of the different proteases, trypsin, Lys-C, and chymotrypsin (Mₐₗₜ) were calculated, and searched in the mass lists of the orphan assayed fragrnents (orphan Mᵢ^{a}; Mⱼ^{a}; and Mₖ^{a}, respectively).

(xii) Only one amino acid change was supported by four different peptides deriving from all three digestion agents (Mᵢ^{a}1=1424.67 Dalton; Mⱼ^{a}1=1937.98 Dalton; Mⱼ^{a}2=2309.16 Dalton and Mₖ^{a}1=2362.24 Dalton). This change comprises the combination of the following changes (as shown in Fig.4 B): amino acids 105-140 were deleted (denoted in italic letters), and the amino acid D (denoted in an underlined letter) was inserted in the joining point. Thus, the assayed protein might be an alternative splice variant of TN10_HUMAN, in which amino acids 105-140 are missing, and the amino acid D appears instead.

Modifications and variations of the present invention, as described above and illustrated in the examples, are possible. It is therefore understood that within the scope of the appended claims, the invention may be realized otherwise than as specifically described.

1. A method for determining the mass of a mass altering moiety, which is present in an assayed peptide and is absent from a corresponding database peptide, or is present in a database peptide and is absent from an assayed peptide, the method comprising:
(i) treating the assayed peptide with a first digestion agent to obtain a first digestion product comprising a plurality of first assayed fragments; and determining the mass spectrum of the digestion product to obtain one or more mass values of the individual first assayed fragments Mᵢ^{a};
(ii) treating the assayed peptide with a further digestion agent to obtain a further digestion product comprising a plurality of further assayed fragments; and determining the mass spectrum of the further digestion product to obtain one or more mass values of the individual further assayed fragments Mⱼ^{a};
(iii) optionally repeating step (ii) according to the number of different further digestion agents, obtaining mass values of the individual further assayed fragments Mₖ^{a}, Mₗ^{a}, Mₘ^{a}, etc.;
(iv) optionally identifying the assayed peptide, in case of a peptide not identified earlier, by a suitable protein identification method;
(v) obtaining masses Mᵢ^{t} of the individual theoretical fragments of the database peptide corresponding to the assayed peptide, which fragments are obtained by the theoretical digestion of said database peptide with said first digestion agent;
(vi) obtaining masses Mⱼ^{t} of the individual theoretical fragments of the database peptide corresponding to the assayed peptide, which fragments are obtained by the theoretical digestion of said database peptide with said further digestion agent;
(vii) comparing each of Mᵢ^{a} with each database value Mᵢ^{t}, to obtain a plurality of differences Dᵢ=Mᵢ^{a}−Mᵢ^{t} and discarding all Dᵢ values lower than a predetermined threshold value to give a plurality of selected differences Dᵢ′;
(viii) comparing each of Mⱼ^{a} with each database value Mⱼ^{t}, to obtain a plurality of differences Dⱼ=Mⱼ^{a}−Mⱼ^{t} and discarding all Dⱼ values lower than a predetermined threshold value to give a plurality of selected differences Dⱼ′;
(ix) comparing selected differences Dᵢ′ and Dⱼ′, preferably comprising overlapping theoretical fragments, and identifying those which are essentially identical; and optionally repeating steps (vi) to (ix), according to the number of different further digestion agents, obtaining selected differences Dₖ′/Dₗ′/Dₘ′, etc. The required mass of said mass altering moiety is thereby defined by said essentially identical Dᵢ′/Dⱼ′ values.

2. The method of claim 1, wherein the mass of the mass altering moiety that was determined is used to determine the identity of the moiety.

3. The method of claim 1, wherein the amino acid sequence shared by said overlapping theoretical fragments is used to determine the identity and/or the location of the mass altering moiety within the amino acid sequence.

4. The method of claim 1, wherein in step (iv) said assayed peptide is identified by any method comprising mass spectrometry, protein sequencing, immunoassay, chromatography, electrophoresis, protein chips, or antibody chips.

5. The method of claim 1, wherein said predetermined threshold value is based on the experimental error of the methods and equipment involved.

6. The method of claim 1, wherein said essentially identical D_{i′}/D_{j′}values defining the mass of said mass altering moiety may differ according to the error of the methods and equipment involved.

7. A method according to claim 1, wherein the mass altering moiety results from a post-translational modification that occurred in-vivo.

8. A method according to claim 1, wherein the mass altering moiety results from a modification that occurred in-vitro during sample preparation.

9. A method according to claim 1, wherein the mass altering moiety results from a mutation.

10. A method according to claim 1, wherein the difference between the assayed and the database peptide is due to a difference in organism strain or species.

11. A method according to claim 1, wherein the mass altering moiety results from alternative splicing.

12. A method according to claim 1, wherein the mass altering moiety results from RNA editing.

13. A method according to claim 1, wherein the difference between the assayed and the database peptide is due to a database error.

14. A method according to claim 1, wherein the difference between the assayed and the database peptide is due to single nucleotide polymorphism (SNPs).

15. A method according to claim 1, wherein the difference between the assayed and the database peptide is due to a signal peptide cleavage.

16. A method according to claim 1, wherein the assayed and the database peptide comprise non-identical, homologue sequences.

17. A method according to claim 1, wherein the mass altering moiety is selected from the group consisting of a sugar moiety, a lipidic moiety, an acyl moiety, an acidic moiety, biotin, a flavin, pyridoxal phosphate, and a moiety added by oxidation of sulphur in the peptide.

18. A method according to claim 1, wherein the mass altering moiety is an amino acid sequence of one or more amino acid residues.

19. A method according to claim 7, wherein the post-translational modification comprises acetylation, amidation, deamidation, farnesylation, formylation, geranylation, hydroxylation, methylation, myristoylation, phosphorylation, and sulphation.

20. A method according to claim 1, wherein the digestion agent is a chemical agent or a proteolytic enzyme.

21. A method according to claim 18, wherein the digestion agent is chosen from the group consisting of cyanogen bromide, trypsin, chymotrypsin, Glu-C, Lys-C, AspN, elastase, and thermolysin.

22. A method for identifying a cleavage altering sequence which is present in an assayed peptide and is absent from a corresponding database peptide, or is present in a database peptide and is absent from an assayed peptide, wherein said cleavage altering sequence alters a cleavage site for at least one digestion agent used in the assay, the method comprising the steps of:
(i) treating the assayed peptide with a first digestion agent to obtain a first digestion product comprising a plurality of first assayed fragments; and determining the mass spectrum of the digestion product to obtain one or more mass values of the individual first assayed fragments Mᵢ^{a};
(ii) treating the assayed peptide with a further digestion agent to obtain a further digestion product comprising a plurality of further assayed fragments; and determining the mass spectrum of the further digestion product to obtain one or more mass values of the individual further assayed fragments Mⱼ^{a};
(iii) optionally repeating step (ii) according to the number of different further digestion agents, obtaining mass values of the individual further assayed fragments Mₖ^{a}, Mₗ^{a}, Mₘ^{a}, etc.;
(iv) optionally identifying the assayed peptide, in case of a peptide not identified earlier, by a suitable protein identification method;
(v) obtaining masses Mᵢ^{t} of the individual theoretical fragments of the database peptide corresponding to the assayed peptide, which fragments are obtained by the theoretical digestion of said database peptide with said first digestion agent;
(vi) obtaining masses Mⱼ^{t} of the individual theoretical fragments of the database peptide corresponding to the assayed peptide, which fragments are obtained by the theoretical digestion of said peptide with said further digestion agent;
(vii) optionally repeating step (vi) according to the number of different further digestion agents, obtaining masses Mₖ^{t} , Mₗ^{t}, Mₘ^{t}, etc., of the individual theoretical fragments.
(viii) comparing each of Mᵢ^{a} with each database value Mᵢ^{t}, to obtain a plurality of differences Dᵢ=Mᵢ^{a}−Mᵢ^{t}; discarding all Mᵢ^{a} and Mᵢ^{t} for which at least one of the Dᵢ values is lower than a predetermined threshold value; and thus identifying orphan Mᵢ^{a} that have no corresponding Mᵢ^{t}, and orphan Mᵢ^{t} that have no corresponding Mᵢ^{a};
(ix) comparing each of Mⱼ^{a} with each database value Mⱼ^{t}, to obtain a plurality of differences Dⱼ=Mⱼ^{a}−Mⱼ^{t}; discarding all Mⱼ^{a} and Mⱼ^{t} for which at least one of the Dⱼ values is lower than a predetermined threshold value; and thus identifying orphan Mⱼ^{a} that have no corresponding Mⱼ^{t}, and orphan Mⱼ^{t} that have no corresponding Mⱼ^{a};
(x) optionally repeating step (ix) according to the number of different further digestion agents, and thus identifying orphan Mₖ^{a}, Mᵢ^{a}, Mₘ^{a}, etc., that have no corresponding Mₖ^{t}, Mₗ^{t, M}ₘ^{t}, etc., and identifying orphan Mₖ^{t}, Mₗ^{t}, Mₘ^{t}, etc. that have no corresponding Mₖ^{a}, Mₗ^{a}, Mₘ^{a}, etc;
(xi) defining a first orphan region as the subset of the amino acid sequences of the database peptide which includes all the theoretical fragments corresponding to orphan Mᵢ^{t} for said first digestion agent; defining a further orphan region as the subset of the amino acid sequences of the database peptide which includes all the theoretical fragments corresponding to orphan Mⱼ^{t} for said further digestion agent; optionally repeating this for further digestion agents Mₖ^{t} etc.; and finally defining a peptide orphan region as the intersection of the first orphan region with all further orphan regions, thus consisting of a subset of sequences of the peptide that were not identified by any of the digestion agents;
(xii) theoretically altering the amino acid sequence of said peptide orphan region, by adding, deleting or changing one or more amino acids thereof, to obtain altered database fragments; and calculating a set of theoretical values of masses Mₐₗₜ of said altered fragments;
(xiii) comparing each Mₐₗₜ with an orphan Mᵢ^{a}; orphan Mⱼ^{a}, orphan Mₖ^{a}, etc. and selecting those Mₐₗₜ of which the difference from an orphan Mᵢ^{a}, Mⱼ^{a}, Mₖ^{a} etc. is smaller than a predetermined threshold value. Mₐₗₜ representing the correct change is selected based on a predetermined criterion, for example, confirmation by the largest number of different digestion agents; and thus identifying the amino acid sequence which is present only in the assayed peptide or in the database peptide as the altered database fragment contributing to said Mₐₗₜ.

23. The method of claim 22, wherein in step (iv) said assayed peptide is identified by any method comprising mass spectrometry, protein sequencing, immunoassay, chromatography, electrophoresis, protein chips, or antibody chips.

24. The method of claim 22, wherein said predetermined threshold value is based on the experimental error of the methods and equipment involved.

25. The method of claim 22, wherein in step (xii), the theoretically alteration of the amino acid sequence, is done based on genomic information.

26. A method according to claim 22, wherein the cleavage altering sequence results from a mutation.

27. A method according to claim 22, wherein the cleavage altering sequence results from a difference in organism strain or species.

28. A method according to claim 22, wherein the cleavage altering sequence results from alternative splicing.

29. A method according to claim 22, wherein the cleavage altering sequence results from RNA editing.

30. A method according to claim 22, wherein the cleavage altering sequence results from a database error.

31. A method according to claim 22, wherein the cleavage altering sequence results from single nucleotide polymorphism (SNPs).

32. A method according to claim 22, wherein the cleavage altering sequence results from a signal peptide cleavage.

33. A method according to claim 22, wherein the assayed and the database peptide comprise non-identical, homologue sequences.

34. A method according to claim 22, wherein the digestion agent is a chemical agent or a proteolytic enzyme.

35. A method according to claim 22, wherein the digestion agent is chosen from the group consisting of cyanogen bromide, trypsin, chymotrypsin, Glu-C, Lys-C, AspN, elastase, and thermolysin.

36. A kit for determining a mass altering moiety and/or cleavage altering sequence of a peptide for use with mass spectroscopy, comprising two or more digestion agents, means for digesting peptides with the agents, and an instruction manual.

37. A kit of claim 36 comprising at least two proteolytic enzymes.
